(19) United States
(12) Patent Application Publication
(10) Pub. No.: US 2006/0258629 A1
(43) Pub. Date: Nov. 16, 2006

(54) Photodynamic therapy for ocular neovascularization

(76) Inventor: William R. Freeman, La Jolla, CA (US)

Correspondence Address: BUCHANAN, INGERSOLL & ROONEY LLP, P.O. BOX 1404, ALEXANDRIA, VA, 22313-1404, US

(21) Appl. No.: 10/531,546

(22) PCT Filed: Oct. 20, 2003
(86) PCT No.: PCT/US03/33365

§ 371 Date: Oct. 21, 2005

Related U.S. Application Data
(60) Provisional application No. 60/419,883, filed on Oct. 18, 2002.

(51) Int. Cl. A61K 38/54; A61K 31/655; A61K 31/555; A61K 31/5415; A61K 31/409; A61N 1/30; A61H 33/00

(52) U.S. Cl. 514/150; 514/185; 514/410; 514/561; 514/224.8; 604/20; 607/86

(57) ABSTRACT

Method, apparatuses and systems are provided for the photodynamic treatment of feeder vessels associated with aberrant choroidal neovasculature.

### CROSS REFERENCE TO RELATED APPLICATIONS

This application claims priority from U.S. Provisional Application Ser. No. 60/419,883, filed Oct. 18, 2002, which is incorporated herein by reference.

### FIELD OF THE INVENTION

The invention relates to methods for treating pathologies associated with ocular neovascularization. The invention further relates to an apparatus useful for treating pathologies associated with ocular neovascularization.

### BACKGROUND

The development of unwanted neovasculature in the choroidal layer (a layer underneath the retina that provides nourishment to the retina) results in a loss of visual acuity. Choroidal neovascularization (CNV) leads to hemorrhage and fibrosis, with resultant visual loss in a number of recognized eye diseases, including ocular histoplasmosis syndrome, myopia, diabetic retinopathy, and age-related macular degeneration (AMD). The “macula” is the center of the retina and is responsible for straight ahead vision, best (reading) vision, and the majority of color vision. The “fovea” is the central part of the macula that provides the sharpest vision.

The most common form of macular degeneration is termed “dry” or involutional macular degeneration and results from the thinning of vascular and other structural or nutritional tissues underlying the retina in the macular region. A more severe form is termed “wet” or exudative macular degeneration. In this form, blood vessels in the choroidal layer break through a thin protective layer between the two tissues. These blood vessels may grow abnormally directly beneath the retina in a rapid uncontrolled fashion, resulting in oozing, bleeding, or eventually scar tissue formation in the macula which leads to severe loss of central vision.

Choroidal neovascularization is generally detected using angiography, e.g., fluorescein angiography, alone or in combination. with indocyanine-green angiography. Once identified, current methods of treatment of wet macular degeneration and proliferative diabetic retinopathy involve destruction of the neovasculature. For example, in laser photocoagulation therapy a surgeon uses a laser to coagulate tissue thereby sealing and destroying leaking blood vessels. Laser photocoagulation involves brief exposures to tiny spots of intense laser light to the area occupied by abnormal blood vessels. The light energy is absorbed and converted to heat energy that cauterizes and destroys the abnormal blood vessels.

The disadvantage of laser photocoagulation is that the procedure destroys cells surrounding the proliferating capillaries, resulting in visual impairment at the treatment site. As a result, this therapy may be repeated only a limited number of times before seriously degrading visual acuity.

Photodynamic therapy (PDT) is another method used to treat these disorders, and involves injection of a photosensitizer, such as verteporfin. The photosensitizer becomes concentrated in the choroidal neovasculature (CNV). Subsequently, the macula or retina is irradiated with low intensity laser light to activate the photosensitizer. It is believed that the photosensitizer absorbs the laser light and releases reactive oxygen intermediates that selectively damage the abnormal blood vessels, while doing less damage to the overlying retina (October 1999, “Archives of Opthalmology”). The FDA recently approved verteporfin for treating wet AMD. However, it is only indicated for patients whose new blood vessels are characterized as “predominantly classic.”

A disadvantage of PDT is that the photosensitizer accumulates in normal choroidal vasculature as well as aberrant neovasculature. The concentration of photosensitizer and number of treatments are generally limited in order to avoid the accumulation of damage to normal tissue. Moreover, aberrant choroidal neovasculature generally recur, forcing the patient to undergo multiple treatments. This cycle of treatment and re-treatment is associated with damage to the normal choriocapillaris vessels and also the retina. Multiple treatments of choroidal neovasculature by PDT is associated with a loss of visual acuity.

Retinal histopathology of patients with choroidal neovascularization has revealed that areas of CNV are usually fed by a few smaller choroidal feeder vessels originating from the choroid or choriocapillaris. The advantage to treating feeder vessels is the possibility that a large CNV complex can be eliminated by closing a small number of feeder vessesls. Further, feeder vessels are generally localized to an area outside the central portion of the macula (i.e., the vessels are “extra-foveal”).

In the past, angiography was used to detect feeder vessels and the CNV associated with them. However, CNV is often more extensive than indicated by conventional angiograms since the vessels are large, have an ill-defined bed, protrude below into the retina and can associate with pigmented epithelium. Recently, advances in retinal imaging using confocal scanning laser ophthalmoscopes have made it possible to image feeder vessels and subsequently treat them.

However, as noted above, laser photocoagulation often results in the destruction of normal tissue even though the targeted feeder vessels are extra-foveal. In general there are multiple feeder vessels that perfuse a CNV complex. Each vessel would need to be targeted for photocoagulation therapy in order to successfully treat the CNV complex thereby increasing the likelihood of destroying normal tissue above and around the targeted vessels. Furthermore, these vessels usually re-open, necessitating additional treatments at the expense of the surrounding tissue. Coagulation either with a hemoglobin absorbing wavelength or with a more penetrating diode 810 nm wavelength often results in incomplete closure requiring multiple treatments or resulting in only partial shut down of the flow into the CNV complex and only a partial readsorption of subretinal fluid. The reason for this is that it is difficult to completely occlude and thrombose a large vessel using thermal lasers, reopening and recanalization is common in these cases and this is responsible for variable results after feeder vessel treatment.

There exists a need for more refined methods of identifying feeder vessels associated with CNV and for occluding such vessels in a manner that minimizes the effect on surrounding normal tissues and maximizes the effect of the treatment.

### SUMMARY OF THE INVENTION

Methods for enhancing photodynamic therapy of target feeder vessels associated with aberrant choroidal neovasculature are provided. An apparatus and system for detecting and treating a target feeder vessel are further provided. The methods and apparatus allow for the closure of subretinal neovascular membranes through the use of photodynamic therapy.

In one embodiment, the invention provides a method for treating an ocular neovascular disease in a patient that includes identifying a feeder vessel associated with aberrant choroidal neovasculature (CNV) and administering a photosensitizer to the patient in an amount effective to facilitate photodynamic therapy (PDT). The photodynamic therapy includes delivering the photosensitizer to the feeder vessel and exposing the photosensitizer to photoactivating light having a wavelength absorbed by the photosensitizer for a time and at an intensity sufficient to inhibit or prevent blood flow from the feeder vessel to the choroidal neovasculature,. The method is useful for treating any condition associated with aberrant neovascularization and is particularly useful for the treatment of intraocular neovascularization associated with age-related macular degeneration.

The photosensitizer is any compound capable of activation by light radiation resulting in the destruction of the surrounding tissue. The photosensitizer can have an absorption spectrum of wavelengths between about 350 nm and 1200 nm. In one aspect, the photosensitizer is administered locally to the patient. In another aspect, the photosensitizer is administered parenterally to the patient.

Aberrant choroidal neovasculature can be identified by image analysis including fluorescein angiography or preferably high speed scanning laser ICG angiography. The identification of a feeder vessel associated with aberrant choroidal neovasculature can occur prior to, contemporaneous with, or subsequent to, administration of a photosensitizer. For example, a photoimaging agent can be administered to the patient and used to identify a feeder vessel, or series of feeder vessels targeted for treatment with a photosensitizer.

In another embodiment, a method of the invention further includes administering an anti-angiogenic factor to the patient prior to, contemporaneous with, or subsequent to, the administration of photodynamic therapy. In one aspect, the photosensitizer is associated with a liposome. In another aspect, the photosensitizer and anti-angiogenic factor are co-associated with a liposome.

In another embodiment, a method of the invention further includes evaluating the treatment response using real-time monitoring of the imaging agent intensity at the site of treatment of the feeder vessel subsequent to administration of photodynamic therapy and optionally re-exposing the site of treatment to light having a wavelength absorbed by the photosensitizer for a time and at an intensity sufficient to further inhibit or prevent blood flow from the feeder vessel to the choroidal neovasculature.

In yet another embodiment, a method of the invention includes treating an ocular neovascular disease in a patient by administering a photoimaging agent to the patient and illuminating the retina of the patient with a fluorescence generating light such that the photoimaging agent in the patient's retina fluoresces and emits fluorescent light; detecting the fluorescent light emitted from the patient's retina; identifying aberrant choroidal neovasculature (CNV); identifying a feeder vessel associated with the aberrant choroidal neovasculature; and administering a photosensitizer to said patient in an amount effective to facilitate photodynamic therapy (PDT).

In another embodiment, the invention provides a system for performing photodynamic therapy on a feeder vessel associated with aberrant choroidal neovasculature in the retina of a patient. The system includes a source of fluorescence generating light configured to illuminate the feeder vessel(s) associated with aberrant neovasculature; a fluorescence detector configured to detect fluorescent light emanating from the feeder vessel; a processor programmed to accumulate, store and analyze fluorescence response data from the fluorescence detector in response to fluorescent light from the feeder vessel; and a source configured to deliver photoactivating light to the patient's retina, wherein the photoactivating light is absorbed by a photosensitizer proximally located in the feeder vessel associated with aberrant neovasculature. In one aspect, the system includes a source of photoactivating light capable of generating a wavelength of about 350 nm to 1200 nm. In another aspect, the system includes an image stabilization source.

In another embodiment, the invention provides an apparatus that includes a scanning laser ophthalmoscope mechanically and operationally associated with a photoactivating light source for delivering light to the retina of an eye for photodynamic therapy.

The invention further provides for a use of a combination of a photosensitizer with photoactivating light in the manufacture of a photoreactive species in vivo for the treatment of a feeder vessel associated with aberrant choroidal neovasculature.

### DETAILED DESCRIPTION

Vascular closure has been observed as one of the consequences of therapeutic PDT which has recently led to the use of PDT in opthalmological disease. The exudative stage of age-related macular degeneration (AMD) with choroidal neovascularization (CNV) commonly leads to rapidly progressive loss of sight. PDT can induce a selective occlusion of CNV via light-induced chemical thrombosis and this effect can be used to effectively treat AMD. Diabetic retinopathy (DR) can be similarly treated. However, destruction of CNV that is not properly targeted or limited to the area requiring treatment can result in undesirable collateral damage to retinal tissue. This, in turn, can lead to reduction in visual acuity. These complications can be addressed by methods, systems and apparatuses that target photoactivation energy to a feeder vessel associated with aberrant choroidal neovasculature.

The methods, systems and apparatuses provided herein have advantages over current techniques by allowing selective destruction of vessels feeding an abnormal plexus of vessels thus destroying the feeder and secondarily the vascular complex without treatment of the macula or other vital structures directly. This also allows for the use of higher photosensitizer and photoactivating light doses to facilitate permanent destruction of the vessel since collateral damage does not extend to the fovea (center of vision). In addition, the risk of re-perfusion of the choroidal neovascular membrane is reduced. Finally, the number of treatments is reduced by the destruction of the target feeder vessels.

In one embodiment, the invention provides a method for treating an ocular neovascular disease in a patient by identifying a feeder vessel associated with aberrant choroidal neovasculature (CNV) and administering a photosensitizer to the patient in an amount effective to facilitate photodynamic therapy (PDT). The photodynamic therapy includes delivering the photosensitizer to the feeder vessel identified and exposing the photosensitizer to photoactivating light having a wavelength absorbed by the photosensitizer for a time and at an intensity sufficient to inhibit or prevent blood flow from the feeder vessel to the choroidal neovasculature.

As used herein, “treatment” refers to any manner in which one or more of the symptoms of a disease or disorder are ameliorated or otherwise beneficially altered. “Amelioration” of the symptoms of a particular disorder by use of a particular photosensitizer or pharmaceutical composition thereof in the methods provided herein refers to any lessening, whether permanent or temporary, lasting or transient that can be attributed to or associated with use of the photosensitizer or pharmaceutical composition thereof in the methods provided herein.

As used herein, an “ocular neovascular disease” is a disease characterized by ocular neovascularization, i.e. the development of abnormal blood vessels in the eye of a patient. Such diseases include, but are not limited to, ischemic retinopathy, intraocular neovascularization, age-related macular degeneration, corneal neovascularization, retinal neovascularization, choroidal neovascularization, diabetic macular edema, diabetic retina ischemia, diabetic retinal edema, and proliferative diabetic retinopathy.

A “patient” refers to any animal having ocular tissue that may be subject to neovascularization. Preferably, the animal is a mammal, which includes, but is not limited to, humans and other primates. The term also includes domesticated animals, such as cows, hogs, sheep, horses, dogs, and cats.

“Photodynamic therapy” or “PDT” refers to any form of phototherapy that uses a light-activated drug or compound, referred to herein as a “photosensitizer” or “photoreactive agent,” to treat a disease or other medical condition characterized by rapidly growing tissue, including the formation of abnormal blood vessels (i.e., angiogenesis). Typically, PDT is a two-step process that involves local or systemic administration of the photosensitizer to a patient followed by activation of the photosensitizer by irradiation with a specific dose of light of a particular wavelength. The term “light” as used herein includes all wavelengths of electromagnetic radiation, including visible light. Preferably, the radiation wavelength is selected to match the. wavelength(s) that excite(s) the photosensitizer. Even more preferably, the radiation wavelength matches the excitation wavelength of the photosensitizer and has low absorption by non-target tissues.

To identify the feeder vessel, a simultaneous injection of fluorescein mixed with ICG can be given and a real time movie like images of the early phase is used to locate feeder vessels. In the “umbrella” type, there is a central hyperfluorescence dot which branches radially into a full fledged net. Most of these vessels arise from the center of the fovea. Direct thermal laser treatment can not be performed on these lesions because the fovea itself would be damaged. However, these vessels may be treated in a manner set forth herein because a non-damaging, low intensity laser light is used to activate the photosensitizing agent localized to the targeted vessel. In the “racquet” type feeder vessel, there is an extrafoveal start of hyperfluorescence which eventually branches into a racquet type distribution forming a net of the membrane.

As used herein, a “photosensitizer” or “photoreactive agent” is a compound or composition that is useful in photodynamic therapy. Such agents are capable of absorbing electromagnetic radiation and emitting energy sufficient to exert a therapeutic effect, e.g., the impairment or destruction of unwanted cells or tissue, or sufficient to be detected in diagnostic applications. The photodynamic therapy according to the invention can be performed using any of a number of photoactive compounds. For example, the photosensitizer can be any chemical compound that collects in one or more types of selected target tissues and, when exposed to light of a particular wavelength, absorbs the light and induces impairment or destruction of the target tissues. Virtually any chemical compound that homes to a selected target and absorbs light may be used in this invention. Preferably, the photosensitizer is nontoxic to the patient to which it is administered and is capable of being formulated in a nontoxic composition. The photosensitizer is also preferably nontoxic in its photodegraded form. Ideal photosensitizers are characterized by a lack of toxicity to cells in the absence of the photochemical effect and are readily cleared from non-target tissues.

Any chemical compound that absorbs light may be used in the methods provided herein. Photosensitizers for use in the methods provided herein include, but are not limited to, indocyanine green, toluidine blue, prodrugs such as aminolevulinic acid, texaphyrins, benzoporphyrins, phenothiazines, phthalocyanines, porphyrins, merocyanines, psoralens, protoporphyrin, methylene blue, Rose Bengal, chlorins such as mono-L-aspartyl chlorin e6, alkyl ether analogs of chlorins, purpurins, bacteriochlorins, pheophorbides, pyropheophorbides, cationic dyes and any other agent that absorbs light in a range of about 500 to about 1100 nanometers. Photosensitizers for use in the methods provided herein are also disclosed in U.S. Pat. Nos. 6,319,273, 6,042,603, 5,913,884, 5,952,366, 5,430,051, 5,567,409, 5,942,534, and U.S. patent application Publication No. 2001/0,022,970, incorporated herein by reference.

The photosensitizer reagents for use in the methods provided herein include but are not limited to porphyrins such as PHOTOPHRIN™ (a QLT, Ltd. brand of sodium porfimer), and FOSCAN™, which is a brand of chlorin. Additional photosensitizers include PURLYTIN™ (tin ethyl etiopurpurin) which is available from Miravant (Santa Barbara, Calif.) and VERTEPORFIN™ (Visudyne™) which is a liposomal benzoporphyrin derivative available from QLT Phototherapeutics (British Columbia, Canada; Ciba Vision, Atlanta, Ga.).

The photosensitizer reagents for use in the methods provided herein include but are not limited to chlorins, bacteriochlorins, phthalocyanines, porphyrins, purpurins, merocyanines, psoralens, benzoporphyrin derivatives (BPD), and porfimer sodium and pro-drugs such as delta-aminolevulinic acid, which can produce photosensitive agents such as protoporlphyrin IX, and other suitable photosensitive compounds including ICG, methylene blue, toluidine blue, texaphyrins, and any other agent that absorbs light in a range of 500 nm to 1100 nm. The photoreactive reagents for use in the methods provided herein include but are not limited to lutetium texaphyrin, marketed as LUTRIN™ (Pharmacyclics, Inc. Sunnyvale, Calif.) or LU-TEX™ (Alcon Laboratories, Fort Worth, Tex.) and bacteriochlorphylls.

Any of the photosensitizers described above can be used in the methods of the invention. Of course, mixtures of two or more photoactive compounds can also be used; however, the effectiveness of the treatment depends on the absorption of light by the photosensitizer so that if mixtures are used, components with similar absorption maxima are preferred.

Methods for activating a sensitizer generally utilize a photoreactive light. As used herein, “photoreactive light” refers to light of sufficient intensity and wavelength to activate the photosensitive agent. For photodynamic therapy, photoreactive light is generally classified as “coherent” light. Coherent light is typically generated from a device commonly known as a laser. However, the present invention encompasses the use of non-coherent photoactivating light as long as the non-coherent light provides the appropriate activating wavelength range for the photosensitizer. As used herein, an “activation wavelength range” is the wavelength range over which the photosensitizer is activated. The photosensitizing agents of the present invention preferably have an absorption spectrum that is within the range of wavelengths between 350 nm and 1200 nm, preferably between about 400 and 900 nm and, most preferably, between 600 and 800 nm.

The photosensitizer is formulated so as to provide an effective concentration to the target ocular tissue. The photosensitizer may be coupled to a specific binding ligand which may bind to a specific surface component of the target ocular tissue or, if desired, by formulation with a carrier that delivers higher concentrations to the target tissue. The nature of the formulation will depend in part on the mode of administration and on the nature of the photosensitizer selected. Any pharmaceutically acceptable excipient, or combination thereof, appropriate to the particular photoactive compound may be used. Thus, the photosensitizer may be administered as an aqueous composition, as a transmucosal or transdermal composition, or in an oral formulation.

The photosensitizer can be administered locally or systemically in any of a wide variety of ways, for example, orally, parenterally (e.g., intravenous, intramuscular, intraperitoneal or subcutaneous injection), topically via patches or implants, or the compound may be placed directly in the eye. The photosensitizing agent can be administered in a dry formulation, such as pills, capsules, suppositories, or patches. The photosensitizing agent also may be administered in a liquid formulation, either alone with water, or with pharmaceutically acceptable excipients, such as are disclosed in Remington's Pharmaceutical Sciences. The liquid formulation also can be a suspension or an emulsion. Suitable excipients for suspensions for emulsions include water, saline, dextrose, glycerol, and the like. These compositions may contain minor amounts of nontoxic auxiliary substances such as wetting or emulsifying agents, antioxidants, pH buffering agents, and the like.

The dose of photosensitizer can vary widely depending a variety of factors, such as the type of photosensitizer; the mode of administration; the formulation in which it is carried, such as in the form of liposomes; or whether it is coupled to a target-specific ligand, such as an antibody or an immunologically active fragment. Other factors which impact the dose of photosensitizing agent include the target cell(s) sought, the patient's weight, and the timing of the light treatment. While various photoactive compounds require different dosage ranges, if green porphyrins are used, a typical dosage is of the range of 0.1-50 mg/M² (of body surface area) preferably from about 1-10 mg/M² and even more preferably about 2-8 mg/M².

The various parameters used for photodynamic therapy in the invention are interrelated. Therefore, the dose of the photosensitizer should be adjusted with respect to other parameters, for example, fluence, irradiance, duration of the light used in photodynamic therapy, and time interval between administration of the dose and the therapeutic irradiation. All of these parameters should be adjusted to produce significant enhancement of destruction of a targeted feeder vessel without significant damage to the eye tissue. The fluence during the irradiating treatment can vary widely, depending on type of tissue, depth of target tissue, and the amount of overlying fluid or blood, but preferably varies from about 50-200 Joules/cm². The irradiance typically varies from about 150-900 mW/cm², with the range between about 150-600 mW/cm² being preferred. However, the present invention provides for the use of higher irradiances which have the advantage of shortening treatment times and increasing the likelihood effecting a targeted feeder vessel.

The optimum time following photosensitizer administration until light treatment can also vary widely depending on the mode of administration, the form of administration, and the specific ocular tissue being targeted. Typical times after administration of the photoactive agent range from about 1 minute to about 2 hours, preferably about 5-30 minutes, and more preferably about 10-25 minutes.

The duration of radiation exposure is preferably between about 1 and 30 minutes, depending on the power of the radiation source. The duration of light irradiation also depends on the fluence desired. For example, for an irradiance of 600 mW/cm², a fluence of 50 J/cm² requires 90 seconds of irradiation; 150 J/cm² requires 270 seconds of irradiation.

The radiation is further defined by its intensity, duration, and timing with respect to dosing with the photosensitive agent (post injection interval). The intensity must be sufficient for the radiation to penetrate skin and/or to reach the target tissues to be treated. The duration must be sufficient to photoactivate enough photosensitive agent to act on the target tissues. Both intensity and duration must, be limited to avoid over-treating the patient. The post injection interval before light application is important, because in general the sooner light is applied after the photosensitive agent is administered, 1) the lower is the required amount of light and 2) the lower is the effective amount of photosensitive agent.

PDT is a method for local and selective tissue or cellular destruction by the action of a particular wavelength of low energy light on the photosensitizing agent. The wavelength of light is selected to correspond to the absorbance spectrum of the photosensitizing agent. The agent capable of being photoactivated is administered to the patient. The agent is transported to feeder vessels associated with aberrant choroidal neovasculature in the retina. Either immediately thereafter, or after an appropriate interval, the agent within the vessel(s) is activated by directing light of the appropriate wavelength to this specific area, and optionally to the surrounding area as previously described. The size of the applied light treatments may be in the range of about 1 mm to about 9 mm.

The selection of the photosensitive agent depends upon several factors. These factors include the site or sites of tissue distribution requiring treatment, the mechanisms of action of the agents themselves, and their specific optimal absorption wavelengths. For example, verteporfin is a synthetic, chlorin-like porphyrin that can be introduced by intravenous injection at a dose of about 1-2 mg/kg. Generally, it is activated by light at 50 J/cm² (absorbance peak of drug) from a non-thermal laser. However, longer exposure times can be used when the present method is practiced because the targeted feeder vessel(s) are usually extrafoveal. In this case, higher dosages of photosensitizer(s) and increased duration and number of treatments can be implemented.

In addition to the use of photosensitizers, the invention further encompasses the use of nanoparticles to deliver heat sufficient to disrupt or ablate a feeder vessel associated with aberrant choroidal neovasculature. Following localization of the nanoparticles to a target feeder vessel, the region is irradiated with a laser, at a wavelength minimally absorbed by the surrounding tissue but preferentially absorbed by, the nanoparticle so as to cause the generation of heat by the nanoparticles sufficient to cause disruption of the feeder vessel but with minimal disruption or ablation of the surrounding tissue. Such wavelength is preferably between 700 nm and 1300 nm and more preferably between 750 nm and 1100 nm. Such preferential absorption results in the nanoparticles absorbing the radiation and converting it to heat with a higher efficiency than radiation is absorbed by the surrounding tissue. Nanoparticles include nanoshells as disclosed in U.S. Pat. No. 6,344,272 (incorporated by reference), metal colloids as disclosed in U.S. Pat. No. 5,620,584 272 (incorporated by reference), fullerenes and derivatized fullerenes, as disclosed in U.S. Pat. Nos. 5,739,376; 6,162,926; 5,994,410, all of which are incorporated by reference, as well as nanotubes including single walled nanotubes, as disclosed in U.S. Pat. No. 6,183,714 (incorporated by reference), which can also be derivatized.

In one implementation, the feeder vessel associated with aberrant choroidal neovasculature is identified by image analysis using a “photoimaging agent.” As used herein, a “photoimaging agent” is a compound or composition that is useful for imaging blood vessels during diagnostic and therapeutic applications. Any method suitable for identifying a target feeder vessel can be used with the current methods. Systems and apparatuses for identifying and treating a targeting feeder vessel are further discussed below. Examples of methods of image analysis include fluorescein angiography and high speed scanning laser ophthalmoscopy (SLO). Generally, feeder vessels are identified by methods which utilize compounds that fluoresce when exposed to a particular wavelength of light. An example of such a photoimaging agent is indocyanine green (ICG).

In one implementation, the feeder vessel associated with aberrant choroidal neovasculature is identified prior to, administration of the photosensitizer by administering a photoimaging agent to the patient. In another implementation, the feeder vessel is identified contemporaneous with administration of the photosensitizer by administering a photoimaging agent to the patient. In yet another implementation, the feeder vessel is identified subsequent to administration of the photosensitizer by administering a photoimaging agent to the patient.

In another embodiment, the invention includes administering an anti-angiogenic factor to the patient prior to,. contemporaneous with, or subsequent to, the administration of photodynamic therapy. It is contemplated that a variety of anti-angiogenic factors may be combined with PDT to treat unwanted CNV. The anti-angiogenesis factor can potentiate the cytotoxity of the PDT thereby enhancing occlusion of the feeder vessel associated with aberrant choroidal neovasculature. In addition, the anti-angiogenesis factor can enhance the selectivity of PDT, for example, by occluding the feeder vessel while at the same sparing the surrounding blood vessels, for example, the retinal and/or surrounding tissue, for example, the retinal epithelium. Furthermore, the anti-angiogenesis factor can be used to reduce or delay the recurrence of the condition.

The term “anti-angiogensis factor” is understood to mean any molecule, for example, a protein, peptide, nucleic acid (ribose nucleic acid (RNA) or deoxyribose nucleic acid (DNA)), peptidyl nucleic acid, organic compound or inorganic compound, that reduces or inhibits the formation of new blood vessels in a mammal.

Numerous anti-angiogenesis factors are well known and thoroughly documented in the art (see, for example, PCT/US99/08335). Examples of anti-angiogenesis factors useful in the practice of the invention, include, for example, anti-VEGF factors including antibodies, peptides and small molecule inhibitors. Additional anti-angiogenic factors include angiostatin, a proteolytic fragment of plasminogen (U.S. Pat. Nos. 5,733,876; 5,837,682; and 5,885,795) including full length amino acid sequences of angiostatin, bioactive fragments thereof, and analogs thereof; endostatin, a proteolytic fragment of collagen XVIII (U.S. Pat. No. 5,854,205) including full length amino acid sequences of endostatin, bioactive fragments thereof, and analogs thereof; and certain antibodies and antigen binding fragments thereof; and peptides that bind preferentially to the epidermal growth factor receptor; antibodies, proteins, peptides and/or nucleic acids that bind preferentially to and neutralize vascular endothelial growth factor antibodies, proteins, and/or peptides that bind preferentially to and neutralize vascular endothelial growth factor receptor; anti-fibroblast growth factor, anti-epidermal growth factor including full length amino acid sequences, bioactive fragments and analogs thereof, and pigment epithelium-derived growth factor, including full length amino acid sequences, bioactive fragments and analogs thereof. Bioactive fragments refer to portions of the intact protein that have at least 30%, more preferably at least 70%, and most preferably at least 90% of the biological activity of the intact proteins. Analogs refer to species and allelic variants of the intact protein, or amino acid replacements, insertions or deletions thereof that have at least 30%, more preferably at least 70%, and most preferably 90% of the biological activity of the intact protein.

In addition, the efficacy and selectivity of the photodynamic therapy method may be enhanced by combining the procedure with an apoptosis-modulating factor. An apoptosis-modulating factor can be any factor, for example, a protein (for example a growth factor or antibody), peptide, nucleic acid (for example, an antisense oligonucleotide), peptidyl nucleic acid (for example, an antisense molecule), organic molecule or inorganic molecule, that induces or represses apoptosis in a particular cell type. For example, it may be advantageous to prime the apoptotic machinery of feeder vessel endothelial cells with an inducer of apoptosis prior to PDT so as to increase their sensitivity to PDT. Endothelial cells primed in this manner are contemplated to be more susceptible to PDT. This approach may also reduce the light dose (fluence) required to achieve feeder vessel closure and thereby decreasing the level of damage on surrounding cells such as RPE. Alternatively, the cells outside the feeder vessel may be primed with an a repressor of apoptosis so as to decrease their sensitivity to PDT. In this approach, the PDT at a particular fluence can become more selective for the targeted feeder vessel associated with CNV.

Apoptosis involves the activation of a genetically determined cell suicide program that results in a morphologically distinct form of cell death characterized by cell shrinkage, nuclear condensation, DNA fragmentation, membrane reorganization and blebbing. It has been suggested that apoptosis is associated with the generation of reactive oxygen species, and that the product of the Bcl-2 gene protects cells against apoptosis by inhibiting the generation or the action of the reactive oxygen species. Apoptosis regulatory gene products include death antagonists (i.e., Bcl-2, BCl-x_{L}) or death agonists (i.e., Bax, Bak).

The apoptosis-inducing factor preferably is a protein, or peptide capable of inducing apoptosis in cells, for example, endothelial cells, disposed in the target feeder vessel. Apoptosis-inducing factors include, for example, constatin, tissue necrosis factor a including bioactive fragments and analogs thereof, cycloheximide, and tunicamycin. Furthermore, other apoptosis-inducing factors may include, for example, anti-sense nucleic acid or peptidyl nucleic acid sequences that reduce or turn off the expression of one or more of the death antagonists (i.e., BCl-2 or Bcl-X_{L}). Antisense nucleotides directed against Bcl-2 have been shown to reduce the expression of Bcl-2 protein in certain lines together with increased phototoxicity and susceptibility to apoptosis during PDT (Zhang et al. (1999) Photochem. Photobiol. 69:582-586).

An increase in efficacy and/or selectivity of the PDT, and/or reduction or delay of recurrence of the CNV can be achieved by (i) administering an anti-angiogenic factor to the patient prior to or concurrent with administration of the photosensitizer, (ii) using a photosensitizer with a targeting molecule that targets the photosensitizer to a feeder vessel associated with aberrant CNV, (iii) administering an apoptosis-modulating factor to the mammal prior to or concurrent with administration of the photosensitizer, (iv) a combination of any two of the foregoing, for example, a combination of the anti-angiogenesis factor and the targeted photosensitizer, a combination of the anti-angiogenesis factor and the apoptosis modulating agent, or a combination of the targeted photosenitizer and the apoptosis modulating agent, or (v) a combination of all three of the foregoing.

The invention further encompasses incorporating the photosensitizer and/or imaging agent and/or anti-angiogenesis agent and/or apoptosis modulating factor in a liposome for delivery to a targeted feeder vessel. The modified liposome can comprise a lipocomplex “package” for delivering a plurality of compounds to a targeted feeder vessel. The modified liposome may be conjugated or associated with a targeting molecule, where the targeting molecule targets the liposome to a feeder vessel associated with a disease state. Such targeting molecules can be antibodies, antibody fragments, receptor binding proteins or other proteins or molecules including growth factors. For example, endothelial cells in new blood vessels express several proteins that are absent or barely detectable in established blood vessels and receptors for certain angiogenic factors like vascular endothelial growth factor (VEGF). In vivo selection of phage peptide libraries have also identified peptides expressed by the vasculature that are organ-specific, implying that many tissues have vascular “addresses.” It is contemplated that a suitable targeting moiety can direct a photosensitizer to the endothelium of a feeder vessel associated with aberrant CNV thereby increasing the efficacy and lowering the toxicity of PDT. For example, liposomal formulations are believed to deliver porphyrins selectively to the low-density lipoprotein component of plasma which, in turn acts as a carrier to deliver the photrosensitizer more effectively to the desired site. By increasing the partitioning of the photosensitizer into the lipoprotein phase of the blood, liposomal formulations can result in a more efficient delivery of the photosensitizer to a feeder vessel associated with choroidal neovasculature. Compositions of porphyrins involving lipocomplexes, including liposomes, are described in U.S. Pat. No. 5,214,036, incorporated herein by reference.

Potential targeting molecules include antibodies for vascular endothelial growth factor receptor (VEGF-2R). Clinical and experimental evidence strongly supports a role for VEGF in ocular neovascularization, particularly ischemia-associated neovascularization. Antibodies to the VEGF receptor (VEGFR-2 also known as KDR) may also bind preferentially to neovascular endothelium. As used herein, the term “antibody” includes, for example, a monoclonal antibody or an antigen binding fragment thereof (i.e., an Fv, Fab, Fab′ or an (Fab′)₂ molecule), a polyclonal antibody or an antigen binding fragment thereof, or a biosynthetic antibody binding site, for example, an sFv (U.S. Pat. Nos. 5,091,513; 5,132,405; 5,258,498; and 5,482,858) that binds specifically to a target ligand. As used herein, the terms binds “specifically” or “preferentially” are understood to mean that the targeting molecule, for example, the antibody, binds to the complementary or target ligand with a binding affinity of at least 10⁵ M⁻¹, and more preferably 10⁷ M⁻¹.

In another embodiment, the invention includes evaluating the treatment response using real-time monitoring of an imaging agent intensity at the site of treatment of the feeder vessel subsequent to administration of photodynamic therapy and optionally re-exposing the site of treatment to light having a wavelength absorbed by the photosensitizer for a time and at an intensity sufficient to further inhibit or prevent blood flow from the feeder vessel to the choroidal neovasculature. In general, effects of the photodynamic therapy as regards reduction of neovascularization subsequent to treating a feeder vessel can be performed using standard fluorescein angiographic techniques at specified periods after treatment. The effectiveness of PDT may also be determined through a clinical evaluation of visual acuity, using means standard in the art, such as conventional eye charts in which visual acuity is evaluated by the ability to discern letters of a certain size, usually with five letters on a line of given size.

Closure of a targeted vessel can usually be observed angiographically by about 40 seconds to a minute in the early frames by hypofluorescence in the treated areas. During the later angiographic frames, a corona of hyperfluorescence begins to appear and then fills the treated area, possibly representing leakage from the adjacent choriocapillaris through damaged retinal pigment epithelium in the treated area. Large retinal vessels in the treated area perfuse following photodynamic therapy, but tend to demonstrate late staining.

In the past, feeder vessels were difficult to identify and generally only observed as extending from a laser scar to recurrent CNV along the perimeter of the laser scar. The present invention addresses this issue by combining scanning laser ophthalmoscopy with a therapeutic photoactivating light source suitable for performing photodynamic therapy on a feeder vessel associated with aberrant choroidal neovasculature. Thus, in another embodiment, the invention provides an apparatus for imaging and treating a feeder vessel associated with choroidal neovasculature including a scanning laser ophthalmoscope comprising a source of fluorescence generating light having a first wavelength suitable for exciting a first photoimaging agent; a source of fluorescence generating light optionally having a second wavelength suitable for exciting a second photoimaging agent; a device for detecting images of the feeder vessel illuminated by the light source(s); photoactivating light source for delivering therapeutic light to the feeder vessel, wherein the photoactivating light is absorbed by a photosensitizer proximally located in the feeder vessel; and opto-mechanical linkage device for coupling the scanning laser ophthalmoscope with the photoactivating light source.

The invention further includes a system for performing photodynamic therapy on a feeder vessel associated with aberrant choroidal neovasculature in the retina of a patient. The system includes a source of fluorescence generating light configured to illuminate the feeder vessel(s) associated with aberrant neovasculature, a fluorescence detector configured to detect fluorescent light emanating from the feeder vessel, a processor programmed to accumulate, store and analyze fluorescence response data from the fluorescence detector in response to fluorescent light from the feeder vessel, and a source configured to deliver photoactivating light to the patient's retina, wherein the photoactivating light is absorbed by a photosensitizer proximally located in the feeder vessel associated with aberrant neovasculature.

The source of fluorescence generating light includes a laser having a characteristic wavelength of about 500 to 800, about 600 to 700, or about 660 to 670 nanometers. Generally, the source of photoactivating light can be a light-emitting diode, laser diode, incandescent light bulb, gas discharge device, polymeric electroluminescent device, halogen bulb, chemical luminescence, vacuum fluorescence, radio frequency excited gas, microwave excited gas, and cold cathode fluorescent tube. The system can further include an image stabilization source which are well known to those skilled in the art of ocular laser surgical techniques. For example, a source for maintaining image stabilization can include digital image processing algorithms can be calibrated to automatically eliminate motion artifacts from acquired images in real-time.

A scanning laser ophthalmoscope (SLO) is a device that can generate images of the retina of a living human eye. In the SLO, scattered light is measured from a focused spot of light as it is scanned across the retina in a raster pattern. Raster scanning is used to move the focused spot across the retina in a raster pattern. The extent of the pattern defines the area of the retina that is being imaged. Positional outputs from the scanning mirrors, combined with scattered intensity information from the light detector, are used to reconstruct the retinal image. Setting the sweep angle on the scanning mirrors controls the field size of the image.

The image is built over time, pixel by pixel, as the spot moves across the retina. An aperture conjugate to (in the image plane of) the desired focal plane in the retina and prior to the light detector can be used to reduce scattered light originating from planes other than the plane of focus. A confocal aperture can be used to do optical slicing, or imaging of different layers in the human retina. Confocal laser scanning technique ensures high spatial resolution, even parallel to the optical axis. Fluorescence light emitted at or near the adjusted focal plane is detected and contributes to the image, while out-of-focus fluorescence light or scattered light is suppressed. Consequently, the two major benefits of the confocal technique are the ability to create three-dimensional information and the very high image contrast.

U.S. Pat. Nos. 5,923,399, 5,943,177 and 5,892,569 to Van de Velde describe different embodiments of a confocal scanning laser ophthalmoscope that is optimized for delivering selective therapeutic laser of various nature to the retina. This includes temporally modulated applications, small threshold continuous applications and applications that use a photosensitizer drug.

Fluorescein angiography (FA) and indocyanine green angiography (ICGA) can be carried out using a confocal scanning laser ophthalmoscope (cSLO). The Heidelberg Retina Tomograph (HRT) and the Heidelberg Retina Angiograph 2 (HRA-2) are examples of confocal scanning ophthalmoscopes useful in the present invention. The Heidelberg Retina Angiograph 2 (HRA-2) uses confocal laser scanning and detection technology to acquire digital fluorescein angiography (FA) and indocyanine green angiography (ICGA) images—separate or simultaneous—with three-dimensional resolution and high frame rate. Lasers with 488 nm and 795 nm wavelength can be used to excite fluorescein and indocyanine green, respectively. Barrier filters at 500 nm and 810 nm provide fluorescence light detection. Red-free and infrared fundus reflectance images can be acquired with lasers at 488 nm and 817 nm wavelength.

The HRA acquires fluorescein angiography images, ICG angiography images, simultaneous fluorescein and ICG angiography images, autofluorescence images, and fundus reflectance images with green and infrared light. In the simultaneous angiography mode, image pairs are acquired at the same time and both live images are displayed side by side on the monitor. Single angiography images can be acquired, as well as series of images at different focal planes, and temporal image sequences.

An apparatus of the invention further includes a opto-mechanical linkage device for coupling the scanning laser ophthalmoscope with the photoactivating light source. “Opto-mechanical linkage device” refers to any device that can be used to combine an SLO with a photoactivating light source. The invention encompasses a scanning laser ophthalmoscope opto-mechanically coupled with multiple external diagnostic or therapeutic light sources through the use of an appropriate beamsplitter, for example. For SLO imaging, an infra-red diode laser 792 nm or 830 nm is preferred. For cSLO psychophysics and microperimetry a visible wavelength e.g. 532 nm or 633 nm laser is convenient. The 532 nm wavelength has a superior visibility, especially during photodynamic therapy employing 664 nm or 689 nm laser light. Photodynamic therapy generally uses different wavelengths of light than those needed to excite a photoimaging agent used to image a feeder vessel. The opto-mechanical linkage device primarily adjusts the position of scanning or imaging light beams to coincide with the external therapeutic light beams in such a way so as to minimize optical distortion and attenuation of the external beams.

In addition to therapeutic and imaging light, an aiming beam of different wavelength is optionally present in an apparatus of the invention. The aiming beam is usually comprised of polarized light.

The method can also employ an image generating instrument such as, but not limited to, a scanning laser ophthalmoscope which allows localization of feeder vessels associated with an abnormal vessel complex in the choroid region of the eye. The imaging device can be linked to a therapeutic laser or similar light source in combination thereby allowing for the accurate localization of the therapeutic light on to the feeder vessel thus enabling selective destruction of the targeted vessel. The doses of light/drug treatment used may be higher than that used for macular treatment of choroidal neovascular membranes. This can be combined with image stabilization technology to allow treatment during. any eye movements thus enabling treatment without anesthetic injection in the region.

It should be noted that the various parameters used for effective, selective photodynamic therapy in the invention are interrelated. Therefore, the dose should also be adjusted with respect to other parameters, for example, fluence, irradiance, duration of the light used in photodynamic therapy, and time interval between administration of the dose and the therapeutic irradiation. All of these parameters should be adjusted to produce significant damage to feeder vessel tissue without significant damage to the surrounding tissue.

Unless defined otherwise, all technical and scientific terms used herein have the same meaning as is commonly understood by one of skill in the art to which this invention belongs. All patents and publications referred to herein are incorporated by reference.

### EXAMPLES

The apparatus and system of the invention include a light source and an imaging device. The light source is selected to provide a wavelength of radiation to interact with a particular photosensitive compound applied to a feeder vessel containing the compound. Suitable light sources comprise, for example, treatment lasers made by Iridex Corporation, Mountain View, Calif. A suitable imaging device is the Heidelberg Retina Tomography (HRT) device or Heidelberg Retina Angiography (HRA) device made by Heidelberg Engineering GmbH, Dossenheim, Germany. However, it is understood that the present invention is not limited to the use of a particular imaging system. Any device capable of identifying a feeder vessel associated with aberrant choroidal neovasculature The only requirement for the imaging device is allows imaging of feeder vessels and can be combined with a photoactivating light source capable of activating a photosensitizer located in the targeted vessel. In addition to the previously identified HRT and HRA systems, additional imaging technologies include indocyanine green (ICG) fundus cameras, ICG video cameras and ICG scanning lasers. Fluorescein or other photoactive imaging compounds can be used in conjunction with the devices discussed above.

Photodynamic therapy with a photosensitizer, such as verteporfin (Visudyne), provides an opportunity to completly close feeder vessels associated with aberrant choroidal neovasculature. These vessels are typically extrafoveal and thus higher light doses (duration) can be used to obtain more extensive destruction of feeder vessels than currently used methods. Feeder vessels in eyes with CNV due to AMD can be identified with high speed SLO ICG and subsequently closed as discussed above. In the present invention, PDT can be performed with higher doses of photosensitizer, increased duration of activating light exposure, increased number of treatments, increased intensity of the activating light, or any combination thereof because the vessels targeted for treatment are generally are extrafoveal in location. This approach is also feasible in eyes without predominantly classic CNV.

The treatment can involve a range of light doses including 50 J/cm², 100 J/cm², 125 J/cm² and 150 J/cm², or higher, delivered extrafoveally over 1, 5, 7, 10, 12, 15, 20, 30 or 60 minutes after the introduction of the photosensitizer to the patient. Generally a predetermined drug dose, for example, 6 mg/m², is used. As previously noted, the invention encompasses the use of a range of dosages, including those that are higher than we normally be used in convention PDT.

The size of the area exposed to therapeutic photoactivating light is also variable as long as the target is a feeder vessel associated with vasculature in need of treatment. For example, a laser spot size of 1,000-2,000 microns can be used at the discretion of the physician performing the procedure. of course, the size of the feeder vessel can be used to determine the area of exposure needed to close the vessel. For example, the size of a feeder vessel is generally in the range of 100 to 500 microns. In the present invention, the laser spot size can be smaller because an activating light source is coupled to an imaging device, such as an HRA or HRT. When using higher light doses, the time of light application necessary for achieving the required light dose can be relatively long (166 seconds for 100 J/cm², 208 s for 125 J/cm² and 249 s for 150 J/cm²).

The treatment can be divided in to consecutive treatments that add-up to the needed exposure time. For example, the photoactivating light treatment be stopped every 50-100 sec, or every 50 J/cm² delivered, and re-commenced about 30 seconds later. Note that 125 J/cm² of light treatment can be interrupted after 2 sets of 83 seconds and completed after the last set of 42 seconds. Once safety parameters have been established for a particular patient, the photoactivating light dose, photosensitizer dosage, photoactivating light exposure time, or number of treatments, can be escalated to affect the closure of a targeted feeder vessel. The following descriptions of dosages and exposure times for performing a method using an apparatus of the invention are exemplary and do not in any limit the invention:

Vials for injection in clear glass vials of 15 mg. Drug dose of about 6 mg of verteporfin/m². Infusion time of about 10 minutes. Light dose of about 50 J/cm², 100 J/cm², 125 J/cm² and 150 J/cM². Light administration for about 15 minutes after end of infusion. Light intensity of about 600 mW/cm². Laser spot size of about 1,000-2,000 microns.

The foregoing description, for purposes of explanation, used specific nomenclature to provide a thorough understanding of the invention. Nevertheless, the foregoing descriptions of the preferred embodiments of the present invention are presented for purposes of illustration and description and are not intended to be exhaustive or to limit the invention to the precise forms disclosed; obvious modifications and variations are possible in view of the above teachings. Accordingly, it is intended that the scope of the invention be defined by the following claims.

What is claimed is:

1. A method for treating an ocular neovascular disease in a patient, said method comprising:
a) identifying a feeder vessel associated with aberrant choroidal neovasculature (CNV); and
b) administering a photosensitizer to the patient in an amount effective to facilitate photodynamic therapy (PDT), wherein the photodynamic therapy comprises:
(i) delivering the photosensitizer to the feeder vessel identified in a); and
(ii) exposing the photosensitizer to photoactivating light having a wavelength absorbed by the photosensitizer for a time and at an intensity sufficient to inhibit or prevent blood flow from the feeder vessel to the choroidal neovasculature.

2. The method of claim 1, wherein the ocular neovascular disease is selected from the group consisting of ischemic retinopathy, intraocular neovascularization, age-related macular degeneration, corneal neovascularization, retinal neovascularization, choroidal neovascularization, diabetic macular edema, diabetic retina ischemia, diabetic retinal edema, and proliferative diabetic retinopathy.

3. The method of claim 2, wherein said neovascular disease is age-related macular degeneration.

4. The method of claim 1, wherein the photosensitizer is selected from the group consisting of indocyanine green, toluidine blue, aminolevulinic acid, texaphyrins, benzoporphyrin derivatives (BPD), phenothiazines, phthalocyanines, porphyrins, chlorins, purpurins, purpurinimides, bacteriochlorins, pheophorbides, pyropheophorbides and cationic dyes.

5. The method of claim 4, wherein the benzoporphyrin derivative is verteporfin.

6. The method of claim 1, wherein the photosensitizer has an absorption spectrum of wavelengths between about 350 nm and 1200 nm.

7. The method of claim 1, wherein the photosensitizer has an absorption spectrum of wavelengths between about 400 nm and 900 nm.

8. The method of claim 1, wherein the photosensitizer has an absorption spectrum of wavelengths between about 600 and 800 nm.

9. The method of claim 1, wherein the photosensitizer is administered locally to the patient.

10. The method of claim 1, wherein the photosensitizer is administered parenterally to the patient.

11. The method of claim 1, wherein the feeder vessel associated with aberrant choroidal neovasculature is identified by image analysis.

12. The method of claim 11, wherein the image analysis is by fluorescein angiography.

13. The method of claim 11, wherein the image analysis is by high speed scanning laser ophthalmoscopy (SLO).

14. The method of claim 1, wherein the feeder vessel associated with aberrant choroidal neovasculature is identified prior to, contemporaneous with, or subsequent to, administration of the photosensitizer by administering a photoimaging agent to the patient, wherein the agent fluoresces when exposed to light.

15. The method of claim 14, wherein the photoimaging agent is a light absorbing compound such as indocyanine green.

16. The method of claim 1, wherein the photoactivating light is coherent light.

17. The method of claim 16, wherein the coherent light is generated by a laser.

18. The method of claim 1, wherein the photoactivating light is non-coherent light.

19. The method of claim 1, further comprising administering an anti-angiogenic factor to the patient prior to, contemporaneous with, or subsequent to, the administration of photodynamic therapy.

20. The method of claim 19, wherein the antiangiogenic factor is an anti-VEGF factor.

21. The method of claim 1, wherein the photosensitizer is associated with a liposome.

22. The method of claim 21, wherein the liposome is targeted to neovascular tissue.

23. The method of claim 1, further comprising:
a) evaluating the treatment response using real-time monitoring of the imaging agent intensity at the site of treatment of the feeder vessel subsequent to administration of photodynamic therapy; and
b) optionally re-exposing the site of treatment to light having a wavelength absorbed by the photosensitizer for a time and at an intensity sufficient to further inhibit or prevent blood flow from the feeder vessel to the choroidal neovasculature.

24. A method for treating an ocular neovascular disease in a patient, the method comprising:
a) administering a photoimaging agent to the patient and illuminating the retina of the patient with a fluorescence generating light such that the photoimaging agent in the patient's retina fluoresces and emits fluorescent light;
b) detecting the fluorescent light emitted from the patient's retina;
c) identifying aberrant choroidal neovasculature (CNV;
d) identifying a feeder vessel associated with the aberrant choroidal neovasculature of c); and
e) administering a photosensitizer to said patient in an amount effective to facilitate photodynamic therapy (PDT), wherein the photodynamic therapy comprises:
(i) delivering the photosensitizer to the feeder vessel identified in d); and
(ii) exposing the photosensitizer to photoactivating light having a wavelength absorbed by the photosensitizer for a time and at an intensity sufficient to inhibit or prevent blood flow from the feeder vessel to the choroidal neovasculature.

25. A system for performing photodynamic therapy on a feeder vessel associated with aberrant choroidal neovasculature in the retina of a patient, the system comprising:
a) a source of fluorescence generating light configured to illuminate the feeder vessel(s) associated with aberrant neovasculature;
b) a fluorescence detector configured to detect fluorescent light emanating from the feeder vessel;
c) a processor programmed to accumulate, store and analyze fluorescence response data from the fluorescence detector in response to fluorescent light from the feeder vessel; and
d) a source configured to deliver photoactivating light to the patient's retina, wherein the photoactivating light is absorbed by a photosensitizer proximally located in the feeder vessel associated with aberrant neovasculature.

26. The system of claim 25 wherein the source of photoactivating light comprises a laser having a characteristic wavelength of about 500 to about 800 nanometers.

27. The system of claim 25, wherein the source of fluorescence generating light comprises a laser having a characteristic wavelength of about 600 to about 700 nanometers.

28. The system of claim 25, wherein the source of fluorescence generating light comprises a laser having a characteristic wavelength of about 660 to about 670 nanometers.

29. The system of claim 25, wherein the source of photoactivating light comprises one of a light-emitting diode, laser diode, incandescent light bulb, gas discharge device, polymeric electroluminescent device, halogen bulb, chemical luminescence, vacuum fluorescence, radio frequency excited gas, microwave excited gas, and cold cathode fluorescent tube.

30. The system of claim 25, further comprising an image stabilization source.

31. An apparatus for imaging and treating a feeder vessel associated with choroidal neovasculature, the apparatus comprising:
a) a scanning laser ophthalmoscope including:
i) a source of fluorescence generating light having a first wavelength suitable for exciting a first photoimaging agent;
ii) a source of fluorescence generating light optionally having a second wavelength suitable for exciting a second photoimaging agent;
iii) a; device for detecting images of the feeder vessel illuminated by the light source of i) or ii);
b) a photoactivating light source for delivering therapeutic light to the feeder vessel, wherein the photoactivating light is absorbed by a photosensitizer proximally located in the feeder vessel; and
c) an opto-mechanical linkage device for coupling the scanning laser ophthalmoscope with the photoactivating light source.

32. The apparatus of claim 31, wherein the first imaging agent is indocyanine green.

33. The apparatus of claim 31, wherein the second imaging agent is fluorescein.

34. The apparatus of claim 31, wherein the first wavelength is about 460 nm to 500 nm.

35. The apparatus of claim 31, wherein the second wavelength is about 780 nm to 820 nm.

36. The apparatus of claim 31, wherein the scanning laser ophthalmoscope is a confocal scanning laser ophthalmoscope.

37. The method of claim 31, wherein the photosensitizer is selected from the group consisting of indocyanine green, toluidine blue, aminolevulinic acid, texaphyrins, benzoporphyrin derivatives (BPD), phenothiazines, phthalocyanines, porphyrins, chlorins, purpurins, purpurinimides, bacteriochlorins, pheophorbides, pyropheophorbides and cationic dyes.

38. The method of claim 36, wherein the benzoporphyrin derivative is verteporfin.

39. The method of claim 31, wherein the photosensitizer has an absorption spectrum of wavelengths between about 350 nm and 1200 nm.

40. The use of a combination of a photosensitizer with photoactivating light in the manufacture of a photoreactive species in vivo for the treatment of a feeder vessel associated with aberrant choroidal neovasculature.
